(19) United States
(12) Patent Application Publication
(10) Pub. No.: US 2024/0299093 A1
(43) Pub. Date: Sep. 12, 2024

(54) SYSTEM, METHOD AND COMPUTER-READABLE STORAGE DEVICE FOR CONTROLLING LASER LIGHT SOURCE OF LITHOTRIPSY DEVICE

(71) Applicant: GYRUS ACMI, INC. D.B.A. OLYMPUS SURGICAL TECHNOLOGIES AMERICA, Westborough, MA (US)

(72) Inventors: Brian M. Talbot, Southborough, MA (US); Kurt G. Shelton, Bedford, MA (US); Anne G. McLoughlin, Exeter, NH (US); Masayasu Chida, Tokyo (JP)

(21) Appl. No.: 18/669,735

(22) Filed: May 21, 2024

Related U.S. Application Data
(63) Continuation of application No. 16/968,801, filed on Aug. 10, 2020.
(60) Provisional application No. 62/628,513, filed on Feb. 9, 2018.

(51) Int. Cl. A61B 18/26; A61B 1/00; A61B 1/015; A61B 1/018; A61B 1/05; A61B 1/06; A61B 1/12; A61B 1/307; A61B 1/31; A61B 17/3207; A61B 18/00; A61B 18/24; A61M 1/00; H01S 3/067; H01S 3/16

(52) U.S. Cl. CPC A61B 18/26; A61B 1/00006; A61B 1/00055; A61B 1/00087; A61B 1/015; A61B 1/018; A61B 1/06; A61B 1/126; A61B 1/307; A61B 1/31; A61B 18/24; A61M 1/74; A61M 1/77; H01S 3/06716; A61B 1/00045; A61B 1/00105; A61B 1/05; A61B 17/320783; A61B 2018/00482; A61B 2018/00505; A61B 2018/00511; A61B 2018/00559; A61B 2018/00642; A61B 2018/00672; A61B 2018/00678; A61B 2018/00696; A61B 2018/00702; A61B 2018/00773; A61B 2018/00982; A61B 2217/005; A61B 2217/007; A61B 2562/0247; A61M 2205/3334; A61M 2205/3344; H01S 3/1616

(57) ABSTRACT

System and methods for controlling a laser light source of a lithotripsy device to fragment or break a target object are disclosed. An exemplary system includes a controller that can perform one or more iterations of a first process and one or more iterations of a second process. The first process includes steps of selecting at least one variable operating parameter of a laser light source of a lithotripsy device; determining a value of each of a plurality of base settings of the at least one variable operating parameter selected; and selecting one of the plurality of base settings based on signals received from the target in response to laser irradiation according to the value of each of the plurality of base settings set. The second process includes controlling the laser light source based on the one of the plurality of base settings selected.

### CROSS-REFERENCE TO RELATED APPLICATIONS

This application is a Continuation of U.S. patent application Ser. No. 16/968,801, filed Aug. 10, 2020, which is a U.S. National Stage filing under 35 U.S.C. § 371 from International Application No. PCT/US2019/017391, filed Feb. 9, 2019, which claims the benefit of U.S. Provisional Application No. 62/628,513, filed on Feb. 9, 2018, the entire contents of which are incorporated herein by reference.

### BACKGROUND

### Field

The invention relates generally to a system, method and computer-readable storage device storing instructions for controlling a laser light source of a lithotripsy device to fragment or break a target object such as a calculus or a stone.

### Description of Related Art

Calculi or stones are hard masses that form in the urinary tract and may cause pain, bleeding, inflection and/or blockage of the flow of urine. Smaller calculi or stones may cause no symptoms and may be passed in urine from the kidneys and through the urinary tract on their own. Larger calculi or stones that do not pass on their own can be removed with lithotripsy.

Lithotripsy can involve use of an endoscope such as a ureteroscope. The endoscope can be inserted through the urethra, into the bladder, up the ureter and into the collecting system of the kidney to reach the calculi or stones. The endoscope can include an imaging device to provide images for guiding the insertion of the endoscope and to visualize the calculi or stones. In some instances, the endoscope can be used with a device inserted through a working channel of the endoscope and out of a distal opening of the working channel to fragment or break a larger calculus or stone into smaller pieces that can be removed with the endoscope or passed in urine. One such device includes an optical fiber for outputting a laser light as an energy source for fragmenting or breaking the calculus or stone.

A calculus or stone can be made of minerals in the urine that form crystals. The calculus or stone can be composed mainly of calcium. However, the calculus or stone can be composed of other substances such as uric acid, cystine, or struvites (a mixture of magnesium, ammonium and phosphate).

The mechanism by which a calculus or stone forms may result in the calculus or stone having a homogenous composition or a heterogeneous composition. A calculus or stone having a homogenous composition is more likely to have a substantially consistent mechanical property throughout the calculus or stone. Such a calculus or stone may be more easily fragmented or broken with laser light having a single set of operating parameters (e.g., energy, peak power, pulse width, average power, and frequency). In contrast, a calculus or stone having a heterogeneous composition is more likely to have a variety of mechanical properties. Such a calculus or stone may be harder to fragment or break with laser light having a single set of operating parameters. Therefore, a need exists for a technique to more effectively and efficiently fragment or break a calculus or stone having a variety of mechanical properties.

### SUMMARY

One embodiment of the invention provides a system comprising: a controller configured to: perform one or more iterations of a first process, wherein in the first process the controller is configured to: select at least one variable operating parameter of a laser light source of a lithotripsy device; determine a value of each of a plurality of base settings of the at least one variable operating parameter selected; and perform, in order, for the each of the plurality of base settings: set the at least one variable operating parameter selected to the value of the each of the plurality of base settings; and control the laser light source to output laser light based on the value of the each of the plurality of base settings set; select one of the plurality of base settings of the at least one variable operating parameter selected; and perform one or more iterations of a second process, wherein in the second process, the controller is configured to control the laser light source based on the one of the plurality of base settings of the at least one variable operating parameter selected. Another embodiment of the invention provides a method comprising: performing one or more iterations of a first process, wherein the first process comprises: selecting at least one variable operating parameter of a laser light source of a lithotripsy device; determining a value of each of a plurality of base settings of the at least one variable operating parameter selected; and performing, in order, for the each of the plurality of base settings: setting the at least one variable operating parameter selected to the value of the each of the plurality of base settings; and controlling the laser light source to output laser light based on the value of the each of the plurality of base settings set; selecting one of the plurality of base settings of the at least one variable operating parameter selected; and performing one or more iterations of a second process, wherein the second process comprises controlling the laser light source based on the one of the plurality of base settings of the at least one variable operating parameter selected.

Another embodiment of the invention provides a computer-readable storage device storing instructions that cause a computer of a controller to: perform one or more iterations of a first process, wherein in the first process the computer is configured to: select at least one variable operating parameter of a laser light source of a lithotripsy device; determine a value of each of a plurality of base settings of the at least one variable operating parameter selected; and perform, in order, for the each of the plurality of base settings: set the at least one variable operating parameter selected to the value of the each of the plurality of base settings; and control the laser light source to output laser light based on the value of the each of the plurality of base settings set; select one of the plurality of base settings of the at least one variable operating parameter selected; and perform one or more iterations of a second process, wherein in the second process, the computer is configured to control the laser light source based on the one of the plurality of base settings of the at least one variable operating parameter selected.

### BRIEF DESCRIPTION OF THE SEVERAL VIEWS OF THE DRAWINGS

FIG. 1 is a schematic illustration of a system including an endoscope device, a lithotripsy device and a control device for fragmenting a calculus in a body lumen, according to an embodiment of the present system.

FIG. 2 is a flowchart of steps performed by a controller according to an embodiment of the present invention.

FIG. 3 is a flowchart of additional steps performed by the controller according to an embodiment of the present invention.

FIG. 4 is a graph illustrating determination of a value of each of a plurality of base settings of at least one variable operating parameter selected according to an embodiment of the present invention.

FIG. 5 is a graph illustrating determination of a value of each of a plurality of optimized settings of at least one variable operating parameter selected according to an embodiment of the present invention.

### DETAILED DESCRIPTION

A system 1 according to one embodiment of the present invention will now be described with reference to the drawings.

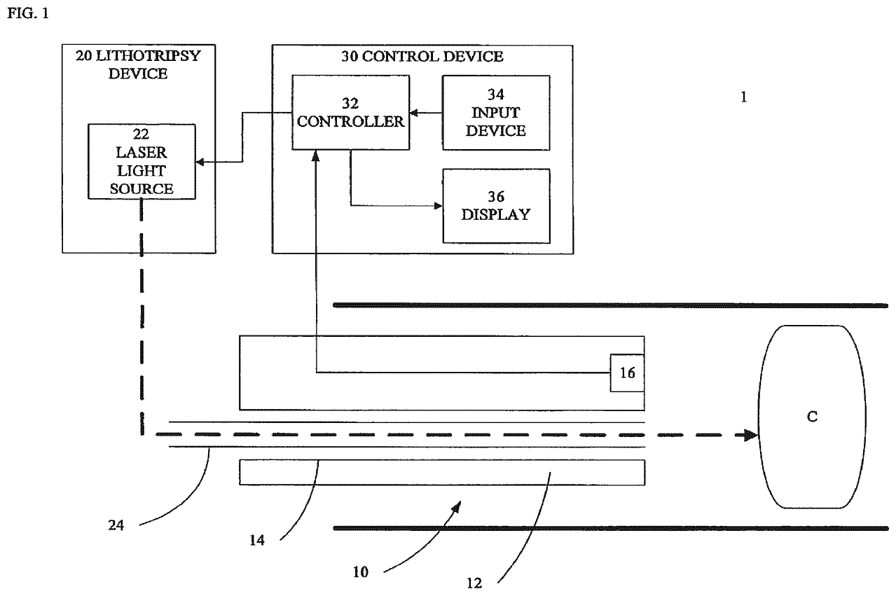

As shown in FIG. 1, the system 1 can include an endoscope device 10, a lithotripsy device 20 and a control device 30, the details of each of which will be described below.

The system 1 can be used in a medical procedure on a body lumen of a subject in order to fragment or break calculi (or stones) in the body lumen. As an example, the body lumen can be a bladder, a ureter or a collecting system of a kidney. However, the system 1 can be used to fragment or break calculi from substantially any body lumen, or from a non-human lumen.

The endoscope device 10 can include an insertion portion 12 having a distal end, where the insertion portion 12 can be sized and provided with sufficient flexibility to be inserted through the urethra, into the bladder, up the ureter and into the collecting system of the kidney to reach a calculus C (or stone). The insertion portion 12 can define a working channel 14 extending through at least a part of the insertion portion 12 to an opening at the distal end of the insertion portion 12. The working channel 14 can be shaped to allow structures such as a treatment instrument or a portion of the lithotripsy device 20 (as described in more detail below) to pass therethrough and past the opening.

The endoscope device 10 can include a light source (not illustrated) and an image sensor 16. The light source of the endoscope device 10 can output a light such as visible light to 25 illuminate the interior of the body lumen and the calculus C. The image sensor 16 can photoconvert returning light incident on an imaging surface of the image sensor 16 into an image signal to be image processed by the control device 30 into an image. By this means, the image sensor 16 and the control device 30 can generate a plurality of images (or a video) over time.

The lithotripsy device 20 can include a laser light source 22 that can output a laser light under the control of the control device 30. The laser light source 22 can be, for example, a holmium (Ho) laser light source, a hulium:YAG (Ho:YAG) laser light source, a neodymium-doped:YAG (nd:YAG) laser light source, a semiconductor laser diode, a potassium-titanyl phosphate crystal (KTP) laser light source, a carbon dioxide (CO2) laser light source, an argon laser light source, an Excimer laser light source, a diode laser light source or another suitable laser light source.

The laser light source 22 can be controlled by the control device 30 to vary one or more operating parameters of the laser light. Operating parameters of the laser light include, but are not limited to energy (E) of the laser light, peak power (Pₚₑₐₖ) of the laser light, pulse width (PW) of the laser light, average power (P_{avg}) of the laser light, and frequency (F) of the laser light.

The operating parameters are related by at least the following equations:

$$E = {P}_{peak} * PW \qquad EQUATION 1$$

$${P}_{avg} = E * F = {P}_{peak} * PW * F . \qquad EQUATION 2$$

The lithotripsy device 20 can further include a light fiber 24 that can be inserted through the working channel 14 of the endoscope device 10 to extend past the opening of the working channel 14. The light fiber 24 can transmit the laser light generated by the laser light source 22 to irradiate the calculus C. Absorbed energy from the laser light can cause the calculus C to fragment or break.

The control device 30 can include a controller 32, an input device 34 and a display 36.

The controller 32 can include a processor comprising hardware, and a storage comprising hardware (e.g., a memory). The functions of the processor may be implemented by respective pieces of hardware or may be implemented by an integrated piece of hardware, for example. The hardware may include one or a plurality of circuit devices (e.g., an integrated circuit (IC)) or one or a plurality of circuit elements (e.g., a resistor, a capacitor, etc) on a circuit board, for example. The processor can be one or more central processing units (CPUs), for example, but this should not be construed in a limiting sense, and various types of processors including a graphics processing unit (GPU) and a digital signal processor (DSP) may be used. The processor may be a hardware circuit with an application-specific integrated circuit (ASIC). The storage comprising hardware may be a semiconductor memory such as a static random-access memory (SRAM) and a dynamic random access memory (DRAM), a register, a magnetic storage device such as a hard disk device, and an optical storage device such as an optical disk device. The storage stores computer-readable instructions, for example. When the instructions are executed by the processor, the functions of the controller 32 described herein are implemented.

The controller 32 can control the imaging device 20 and the lithotripsy device 20 by the techniques described in detail below.

The input device 34 can include a device that can receive inputs from a user. The input device 34 can include a pointing device, a touch screen, a keypad and non-tactile entities such as voice control.

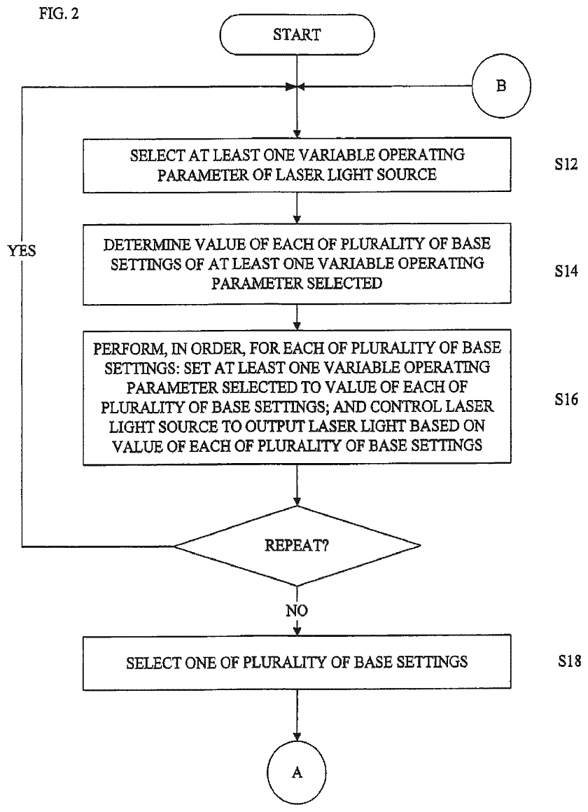

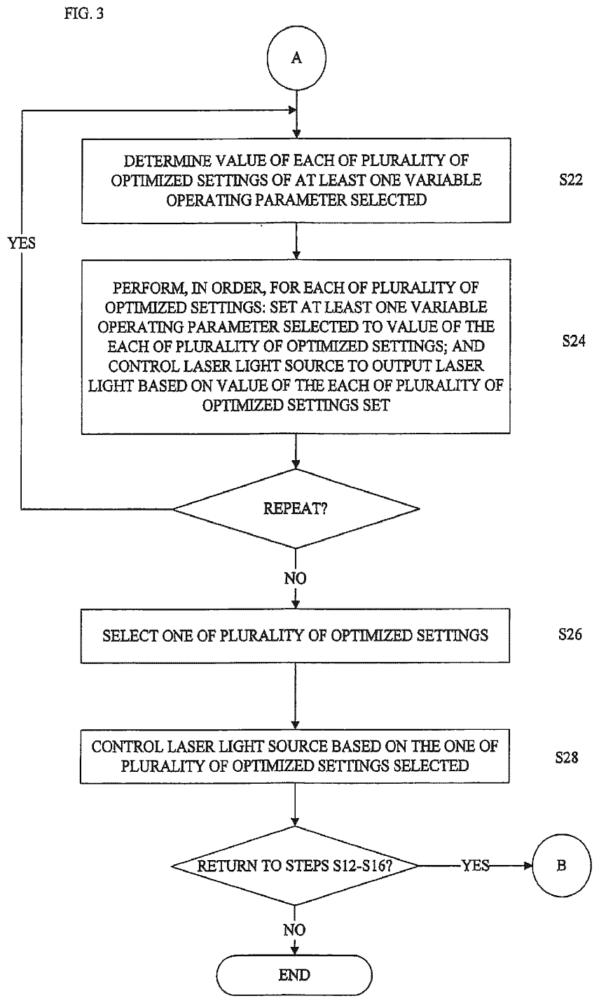

Processes that can be performed by the controller 32 in cooperation with the input device 34, the display 36, the endoscope device 10 and the lithotripsy device 20 are described in detail below with reference to FIGS. 2 and 3.

As shown in FIG. 2, the controller 32 can perform one or more iterations of a first process.

Each iteration of the first process can include steps S12 to S16.

At step S12, the controller 32 can select at least one variable operating parameter of the laser light source 22 of the lithotripsy device 20. The at least one variable operating parameter of the laser light source 22 can include one or more of, for example, energy (E) of the laser light output by the laser light source 22, peak power (Pₚₑₐₖ) of the laser light output by the laser light source 22, pulse width (PW) of the laser light output by the laser light source 22, average power (P_{avg}) of the laser light output by the laser light source 22, and frequency (F) of the laser light output by the laser light source 22.

At Step S14, the controller 32 can determine a value of each of a plurality of base settings of the at least one variable operating parameter selected.

At Step S16, the controller 32 can perform, in order, for the each of the plurality of base settings, setting the at least one variable operating parameter selected to the value of the each of the plurality of base settings, and controlling the laser light source 22 to output laser light at the calculus C based on the value of the each of the plurality of base settings set.

After performing one or more iterations of the first process, the controller 32 can, at Step S18, select one of the plurality of base settings of the at least one variable operating parameter selected.

Next, as shown in FIG. 3, the controller 32 can perform one or more iterations of a 30 second process.

Each iteration of the second process can include Steps S22 and S24.

At Step S22, the controller 32 can determine a value of each of a plurality of optimized settings of the at least one variable operating parameter selected based on the one of the plurality of base settings of the at least one variable operating parameter selected.

At Step S24, the controller 32 can perform, in order, for the each of the plurality of optimized settings, setting the at least one variable operating parameter selected to the value of the each of the plurality of optimized settings, and controlling the laser light source 22 to output laser light at the calculus C based on the value. of the each of the plurality of optimized settings set.

After performing one or more iterations of the second process, the controller 32 can, at Step S26, select one of the plurality of optimized settings of the at least one variable operating parameter selected based on the change in the characteristic of the target object determined.

Next, at Step S28, the controller 32 can control the laser light source 22 to output laser light at the calculus C based on the one of the plurality of optimized settings of the at least one variable operating parameter selected.

Following Step S28, the controller 32, together with the image sensor 16, can generate one or more images of the calculus C that has been treated with the laser light having operating parameters optimized by the first and second processes. The controller 32 can control the display 36 to display the one or more images to allow the user, viewing the one or more images, to determine whether one layer of the calculus C having one mechanical property has been removed by being fragmented or broken to reveal another layer of the calculus C having a different mechanical property that can be more efficiently fragmented or broken under a different optimized setting.

Following Step S28, the input device 34 can receive one or more inputs from the user, and output one or more instructions to the controller 32 based on the one or more inputs. Further, the controller 32 can determine, based on the one or more instructions, whether the user has instructed to return to Steps S12 to S16 for determination of the values of each of a plurality of base settings that are more suitable for fragmenting or breaking the another layer of the calculus C. If returning to Steps S12 to S16 is instructed, the controller 32 can execute Steps S12 to S16 again. If returning to Steps S12 to S16 is not desired, the above-described process ends.

Next, details of Steps S12-S28 will be described by way of examples.

### First Example

The details of Steps S12-S28 will be described below by way of a first example.

At Step S12, the input device 34 can receive one or more inputs from the user and output one or more instructions corresponding to the one or more inputs to the controller 32. At Steps S12 and S14, the controller 32 can receive the one or more instructions from the input device 34, select the peak power Pₚₑₐₖ (or the pulse width PW) of the laser light output by the laser light source 22 as the variable operating parameter based on the one or more instructions received from the input device 34, and determine a value of each of a plurality of base settings of the peak power Pₚₑₐₖ (or the pulse width PW) of the laser light.

The controller 32 can determine the value of each of the plurality of base settings of the peak power Pₚₑₐₖ of the laser light based on the one or more instructions received from the input device 34.

The one or more instructions received from the input device 34 can indicate a first range (that is, an upper limit and a lower limit) of values of the peak power Pₚₑₐₖ of the laser light. The controller 32 can then determine the value of each of the plurality of base settings of the peak power Pₚₑₐₖ of the laser light that falls within the first range of the values of the peak power Pₚₑₐₖ of the laser light indicated by the one or more instructions. Moreover, the controller 32 can determine the value of the each of the plurality of base settings of the peak power Pₚₑₐₖ to be evenly distributed within the first range of values of the peak power Pₚₑₐₖ.

In a modification of the first example, the input device 34 can receive one or more inputs indicating values of each of the plurality of base settings of the peak power Pₚₑₐₖ within the first range, and output one or more instructions corresponding to the one or more inputs to the controller 32. The controller 32 can then determine the value of each of the plurality of base settings of the peak power Pₚₑₐₖ of the laser light based on the one or more instructions received from the input device 34.

At Step S16, the controller 32 can perform, in order, for each of the plurality of base settings, setting the peak power Pₚₑₐₖ of the laser light to the value of the each of the plurality of base settings, and controlling the laser light source 22 to output the laser light having the peak power Pₚₑₐₖ set to the value of the each of the plurality of base settings towards the calculus C to try to or begin to fragment or break the calculus C.

After performing a first iteration of the first process (including Steps S12-S16), the controller 32 can perform a second or subsequent iteration of the first process. In the second or subsequent iteration of the first process, the controller 32 can select another variable operating parameter such as the frequency F of the laser light output by the laser light source 22 and proceed through Steps S14 and S16 based on the selection of the frequency F of the laser light as the variable operating parameter of the laser light.

At Step S18, the controller 32, together with the image sensor 16, can generate one or more images of the calculus C having been treated by the different laser lights having peak power Pₚₑₐₖ set at the value of the each of the plurality of base settings. The controller 32 can control the display 36 to display the one or more images generated to allow the user to judge the efficacy of each of the different laser lights to fragment or break the calculus C.

Further, at Step S18, the input device 34 can receive one or more inputs from the user (having reviewed the one or more images of the calculus C displayed on the display 36) and output one or more instructions corresponding to the one or more inputs to the controller 32. The controller 32 can then select one of the plurality of base settings of the peak power Pₚₑₐₖ of the laser light in accordance with the user's one or more inputs. The one of the plurality of base settings of the peak power Pₚₑₐₖ selected indicates the user's determination, based on the one or more images of the calculus C displayed on the display 36, that the one of the plurality of base settings of the peak power Pₚₑₐₖ is the most effective amongst the plurality of base settings for fragmenting or breaking the calculus C.

At Step S22, the controller 32 can determine a value of each of a plurality of optimized settings of the peak power Pₚₑₐₖ of the laser light based on the one of the plurality of base settings selected in Step S18.

Here, the controller 32 can set a second range (that is an upper limit and a lower limit) of the peak power Pₚₑₐₖ of the laser light, where the second range is smaller than the first range established in Step S14. The controller 32 can then determine the value of each of the plurality of optimized settings of the peak power Pₚₑₐₖ of the laser light that falls within the second range of the peak power Pₚₑₐₖ of the laser light. Moreover, the controller 32 can determine the value of the each of the plurality of optimized settings of the peak power Pₚₑₐₖ to be evenly distributed within the second range of the peak power Pₚₑₐₖ.

At Step S24, the controller 32 can perform, in order for each of the plurality of optimized settings, setting the peak power Pₚₑₐₖ of the laser light to the value of the each of the plurality of optimized settings of the peak power Pₚₑₐₖ, and controlling the laser light source 22 to output laser light based on the value of the each of the plurality of optimized settings set.

At Step S26, the controller 32, together with the image sensor 16, can generate one or more images of the calculus C having been treated by the different laser lights having peak power Pₚₑₐₖ set at the value of the each of the plurality of optimized settings. The controller 32 can control the display 36 to display the one or more images generated to allow the user to judge the efficacy of the different laser lights to fragment or break the calculus C.

Further, at Step S26, the input device 34 can receive one or more inputs from the user (having reviewed the one or more images of the calculus C displayed on the display 36) and output one or more instructions corresponding to the one or more inputs to the controller 32. The controller 32 can then select one of the plurality of optimized settings of the peak power Pₚₑₐₖ of the laser light in accordance with the user's one or more inputs. The one of the plurality of optimized settings of the peak power Pₚₑₐₖ selected indicates the user's determination, based on the one or more images of the calculus C displayed on the display 36 that the one of the plurality of optimized settings of the peak power Pₚₑₐₖ is the most effective amongst the plurality of optimized settings for fragmenting or breaking the calculus C.

At Step S28, the controller 32 can further control the laser light source 22 based on the one of the plurality of optimized settings of the peak power Pₚₑₐₖ of the laser light selected to more effectively and efficiently fragment or break the calculus C.

### Second Example

The details of Steps S12-S28 will be described below by way of a second example.

The second example differs from the first example in that at Steps S12 and S14, the controller 32 can receive the one or more instructions from the input device 34, select a plurality of variable operating parameters (instead of a single variable operating parameter as in the first example) based on the one or more instructions received from the input device 34, and determine a value of each of a plurality of base settings of the plurality of operating parameters of the laser light.

Referring to EQUATION 1 discussed above, the controller 32 can select the peak power Pₚₑₐₖ and the pulse width PW of the laser light as the plurality of variable operating parameters.

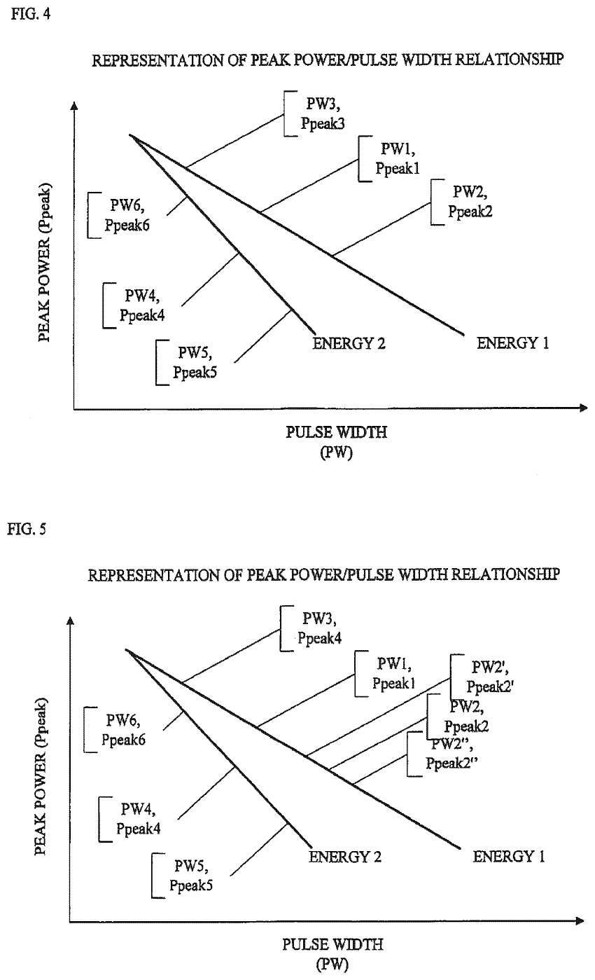

Further, as shown in FIG. 4, the controller 32 can determine a value of each of a plurality of base settings of the peak power Pₚₑₐₖ and the pulse width PW of the laser light. Specifically, the controller 32 can determine combinations of values of the peak power Pₚₑₐₖ and the pulse width PW which will allow the energy E of the laser light to be constant. For example, the controller 32 can determine, for a first base setting, the value of the peak power Pₚₑₐₖ to be Pₚₑₐₖ₁ and the value of the pulse width PW to be PW1, where the laser light output under the operating parameters of the peak power Pₚₑₐₖ having a value of Pₚₑₐₖ₁ and the pulse width PW having a value of PW1 will have a predetermined energy Energy1.

The controller 32 can further determine for a second base setting, the value of the peak power Pₚₑₐₖ to be Pₚₑₐₖ₂ and the value of the pulse width PW to be PW2, where Pₚₑₐₖ₂ is less than Pₚₑₐₖ₁, where PW2 is greater than PW1, and where the laser light output under the operating parameters of the peak power Pₚₑₐₖ having a value of Pₚₑₐₖ₂ and the pulse width PW having a value of PW2 will have the same predetermined energy Energy1.

The controller 32 can further determine for a third base setting, the value of the peak power Pₚₑₐₖ to be Pₚₑₐₖ₃ and the value of the pulse with PW to be PW3, where Pₚₑₐₖ₃ is greater than Pₚₑₐₖ₁, where PW3 is less than PW1, and where the laser light output under the operating parameters of the peak power Pₚₑₐₖ having a value of Pₚₑₐₖ₃ and the pulse width PW having a value of PW3 will have the same predetermined energy Energy1.

In a second or subsequent iteration of the first process, the controller 32 can determine combinations of values of the peak power Pₚₑₐₖ and the pulse width PW of the laser light that will allow the laser light to have a predetermined energy Energy2, where Energy2 is different from Energy1.

In the second or subsequent iteration of the first process, the controller 32 can also select other variable operating parameters. For example, the controller 32 can, referring to EQUATION 2 discussed above, select the frequency F and the pulse width PW as the plurality of variable operating parameters.

Further, referring to Equation 2, the controller 32 can determine a value of each of a plurality of base settings of the frequency F and the pulse width PW of the laser light. Specifically, the controller 32 can determine combinations of values of the frequency F and the pulse width PW which will allow the average power P_{avg} of the laser light to be constant. In this example, referring to EQUATION 2, the controller 32 maintains the peak power Pₚₑₐₖ of the laser light to be unchanged based on evidence that high peak powers are more effective at fragmenting or breaking the calculus C.

The second example is similar to the first example in that the controller 32 can determine the value of the each of the plurality of base settings of the plurality of operating parameters to be within the first range of values. Specifically, the controller 32 can determine Pₚₑₐₖ, Pₚₑₐₖ₂ and Pₚₑₐₖ₃ to be within a first range of peak power values, and determine PW1, PW2 and PW3 to be within first range of pulse width values.

The second example further differs from the first example in that at Step S22 the controller 32 can determine a value of each of a plurality of optimized settings of the plurality of operating parameters of the laser light.

Further, as shown in FIG. 5, the controller 32 can, referring to EQUATION 1 discussed above, determine the value of each of a plurality of optimized settings of the peak power Pₚₑₐₖ and the pulse width PW of the laser light based on the selection of the base setting having values Pₚₑₐₖ₂ and PW2 in Step S18. Specifically, the controller 32 can determine combinations of values of the peak power Pₚₑₐₖ and the pulse width PW which will allow the energy E of the laser light to be constant. For example, the controller 32 can determine, for a first optimized setting, the value of the peak power Pₚₑₐₖ to be Pₚₑₐₖ₂ and the value of the pulse width PW to PW2, where the laser light output under the operating parameters of the peak power Pₚₑₐₖ having a value of Pₚₑₐₖ₂ and the pulse width PW having a value of PW2 will have the predetermined energy Energy1.

The controller 32 can further determine for a second optimized setting, the value of the peak power Pₚₑₐₖ to be Pₚₑₐₖ₂ and the value of the pulse width PW to be PW2′ where Pₚₑₐₖ₂ is greater than Pₚₑₐₖ₂, where PW2′ is less than PW2, and where the laser light output under the operating parameters of the peak power Pₚₑₐₖ having value of P_{peak2′} and the pulse with PW having a value of PW2′ will have the same predetermined energy Energy1.

The controller 32 can further determine for a third optimized setting, the value of the peak power Pₚₑₐₖ to be P_{peak2″} and the value of the pulse width PW to be PW2″ where P_{peak2″} is less than Pₚₑₐₖ₂, where PW2″ is greater than PW2, and where the laser light output under the operating parameters of the peak power Pₚₑₐₖ having value of P_{peak2″} and the pulse with PW having a value of PW2″ will have the same predetermined energy Energy1.

### Third Example

The details of Steps S12-S28 will be described below by way of a third example.

The third example differs from the second example in that one or more of Steps S12, S14, S18, S22 and S26 can be performed in accordance with a characteristic of the calculus C detected by a sensor.

In Steps S12 and S14, the controller 32 can, together with the image sensor 16, generate one or more images of the calculus C. The controller 32 can further process the one or more images of the calculus C to detect one or more characteristics of the calculus C. The one or more characteristics of the calculus C can include, but is not limited to, the size of the calculus C, the color of the calculus C, and the outer geometry of the calculus C. Further, the controller 32 can select the at least one variable operating parameters of the laser light based on the one or more characteristics of the calculus C detected. Still further, the controller 32 can determine a value of each of a plurality of base settings of the at least one variable operating parameter selected based on the one or more characteristics of the calculus C detected. An an example, in Step S12, the controller 32 can, in response to determining the size of the calculus C to be above a predetermined size set energy E to a higher predetermined value, select peak power Pₚₑₐₖ and pulse width PW to be the at least one variable operation parameter, and determine the value of the of the plurality of base settings that satisfy Equation 1.

The memory of the controller 32 can also store predetermined relationships between one or more characteristics and corresponding at least one variable operating parameters of the laser light. The controller 32 can then consider the one or more characteristics of the calculus C detected in view of the stored predetermined relationships in order to select the at least one variable operating parameter in Step S12 and to determine the value of the each of the plurality of base settings of the at least one variable operating parameter selected in Step S14.

In Step S18, the controller 32 can, together with the image sensor 16, generate one or more images of the calculus C after treating the calculus C with the laser light according to Steps S12-S16. The controller 32 can further process the one or more images of the calculus C to detect one or more characteristics of the calculus C. The one or more characteristics of the calculus C can include, but is not limited to, a change in the size of the calculus C, a change in the color of the calculus C, and a change in the outer geometry of the calculus C. The controller 32 can further select one of the plurality of base settings based on the one or more characteristics of the calculus C detected. As an example, in Step S18, the controller 32 can determine an image showing a greatest reduction in size of the calculus C, determine the one of the plurality of base settings resulting in the greatest reduction in size of the calculus C and select the one of the plurality of base settings determined.

In Step S22, the controller 32 can, together with the image sensor 16, generate one or more images of the calculus C. The controller 32 can further process the one or more images of the calculus C to detect one or more characteristics of the calculus C. The one or more characteristics of the calculus C can include, but is not limited to, the size of calculus C, the color of the calculus C, and the outer geometry of the calculus C. Further, the controller 32 can determine a value of each of a plurality of optimized settings of the at least one variable operating parameter selected based on the one or more characteristics of the calculus C detected.

In Step S26, the controller 32 can, together with the image sensor 16, generate one or more images of the calculus C after treating the calculus C with the laser light according to Steps S22 and S24. The controller 32 can further process the one or more images of the calculus C to detect one or more characteristics of the calculus C. The one or more characteristics of the calculus C can include, but is not limited to, a change in the size of the calculus C, a change in the color of the calculus C, and a change in the outer geometry of the calculus C. The controller 32 can further select one of the plurality of optimized settings based on the one or more characteristics of the calculus C detected. As an example, in Step S26, the controller 32 can determine an image showing a greatest reduction in size of the calculus C, determine the one of the plurality of optimized settings resulting in the greatest reduction in size of the calculus C and select the one of the plurality of optimized settings determined.

After Step S28, the controller 32 can, together with the image sensor 16, generate one or more images of the calculus C after treating the calculus C with the laser light according to Step S28. The controller 32 can further process the one or more images of the calculus C to detect one or more characteristics of the calculus C. The controller 32 can further determine whether to return to Steps S12-S16 based on the one or more characteristics of the calculus C detected. As an example, the controller 32 can further determine to return to Steps S12-S16 based on a determination that a change in the size of the calculus C is at or below a predetermined threshold, or that the a change in the color of the calculus Cis at or below a predetermined amount.

In the description provided above, the functions of generating and processing images, and the functions of controlling the laser light source 22 are described as being performed by the controller 32. However, it is understood that the functions of generating and processing images and the functions of controlling the laser light source 22 can be performed by separate controllers in communication with each other.

Another embodiment of the present invention includes a method performed by the controller 32 described above.

Still, another embodiment of the present invention includes a computer-readable storage device storing instructions that can cause a processor comprising hardware of the controller 32 to perform the functions described above.

In the technique described above, after Step S18, one of the plurality of base settings of the peak power Pₚₑₐₖ (or another variable operating parameter) of the laser light output by the laser light source 22 that is more effective for fragmenting or breaking the calculus C having a particular mechanical property corresponding the one of the plurality of base settings is selected to more effectively fragment or break the calculus C having the particular mechanical property. Such a selection in Step S18 represents an improvement over conventional lithotripsy techniques. Further, after Step S28, selecting one of the plurality of optimized settings allows for even more effective fragmenting or breaking of the calculus C. Such a selection in Step S28 represents an additional improvement over conventional lithotripsy techniques.

While embodiments of the present invention have been described, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A medical laser system, comprising:
a laser source configured to generate laser light to irradiate a target in a patient;
an image sensor configured to generate an image of the target; and
a controller configured to:
perform one or more iterations of a first process comprising steps of selecting at least one variable operating parameter of the laser source, determining a value of each of a plurality of base settings of the at least one variable operating parameter selected, and selecting a base setting from the plurality of base settings;
determining a characteristic of the target based at least in part on the generated image of the target;
based at least in part on the determined characteristic of the target, perform one or more iterations of a second process to optimize the selected base setting, the second process including steps of determining a value of each of a plurality of optimized settings relative to the selected base setting of the at least one variable operating parameter selected, and selecting an optimized setting from the plurality of optimized settings; and
control the laser source to output laser light in accordance with the selected optimized setting.

2. The medical laser system of claim 1, wherein the characteristic of the target detected from the generated image includes at least one of:
a size or a change in size of the target;
a color or a change in color of the target; or
an outer geometry or a change in outer geometry of the target.

3. The medical laser system of claim 1, wherein the selected at least one variable operating parameter includes at least one of:
energy (E) of the laser light output by the laser source;
peak power (Pₚₑₐₖ) of the laser light output by the laser source;
pulse width (PW) of the laser light output by the laser source;
average power (P_{avg}) of the laser light output by the laser source; or
frequency (F) of the laser light output by the laser source.

4. The medical laser system of claim 3, wherein the selected at least one variable includes the peak power (Pₚₑₐₖ) and the pulse width (PW) of the laser light output by the laser source.

5. The medical laser system of claim 3, wherein the selected at least one variable includes the peak power (Pₚₑₐₖ), the pulse width (PW), and the frequency of the laser light output by the laser source.

6. The medical laser system of claim 1, wherein selecting the at least one variable operating parameter of the laser source or determining the value of each of the plurality of base settings of the at least one variable operating parameter selected in the one or more iterations of the first process is based at least in part on the determined characteristic of the target.

7. The medical laser system of claim 6, wherein the determined characteristic of the target includes a size of the target determined to be above a predetermined size,
wherein the controller is configured to, in response to the size of the target being above the predetermined size, set energy E to a predetermined value, and select peak power Pₚₑₐₖ and pulse width PW to be the at least one variable operation parameter, and determine the value of the of the plurality of base settings that satisfy E=P_{Peak}*PW.

8. The medical laser system of claim 1, wherein the controller is configured to store in a memory a predetermined relationships between one or more characteristics of target and corresponding at least one variable operating parameters of the laser light,
wherein selecting the at least one variable operating parameter of the laser source or determining the value of each of the plurality of base settings of the at least one variable operating parameter selected in the one or more iterations of the first process is based at least in part on the stored predetermined relationship.

9. The medical laser system of claim 1, wherein selecting the base setting from the plurality of base settings in the one or more iterations of the first process is based at least in part on the characteristic of the target in response to the laser light produced by the laser source and incident on the target in accordance with the value of the each of the plurality of base settings.

10. The medical laser system of claim 9, wherein the selected base setting corresponds to the laser light produced in accordance therewith results in a relatively higher reduction in size of the target than other of the plurality of base settings.

11. The medical laser system of claim 1, wherein determining the value of each of the plurality of optimized settings of the at least one variable operating parameter selected in the one or more iterations of the second process is based at least in part on the determined characteristic of the target.

12. The medical laser system of claim 1, wherein selecting the optimized setting from the plurality of optimized settings in the one or more iterations of the second process is based at least in part on the determined characteristic of the target in response to the laser light produced by the laser source and incident on the target in accordance with the value of the each of the plurality of optimized settings.

13. The medical laser system of claim 12, wherein the selected optimized setting corresponds to the laser light produced in accordance therewith results in a relatively higher reduction in size of the target than other of the plurality of optimized settings.

14. A method comprising:
generating laser light from a laser source to irradiate a target in a patient;
generating an image of the target using an image sensor;
performing one or more iterations of a first process including steps of selecting at least one variable operating parameter of a laser source, determining a value of each of a plurality of base settings of the at least one variable operating parameter selected, and selecting a base setting from the plurality of base settings;
determining a characteristic of the target based at least in part on the generated image of the target;
based at least in part on the determined characteristic of the target, performing one or more iterations of a second process to optimize the selected base setting, the second process including steps of determining a value of each of a plurality of optimized settings relative to the selected base setting of the at least one variable operating parameter selected, and selecting an optimized setting from the plurality of optimized settings; and
outputting laser light from the laser source in accordance with the selected optimized setting.

15. The method of claim 14, wherein the characteristic of the target detected from the generated image includes at least one of:
a size or a change in size of the target;
a color or a change in color of the target; or
an outer geometry or a change in outer geometry of the target.

16. The method of claim 14, wherein the selected at least one variable operating parameter includes at least one of:
energy (E) of the laser light output by the laser source;
peak power (Pₚₑₐₖ) of the laser light output by the laser source;
pulse width (PW) of the laser light output by the laser source;
average power (P_{avg}) of the laser light output by the laser source; or
frequency (F) of the laser light output by the laser source.

17. The method of claim 14, wherein selecting the at least one variable operating parameter of the laser source or determining the value of each of the plurality of base settings of the at least one variable operating parameter selected in the one or more iterations of the first process is based at least in part on the determined characteristic of the target.

18. The method of claim 14, further comprising storing in a memory a predetermined relationships between one or more characteristics of target and corresponding at least one variable operating parameters of the laser light,
wherein selecting the at least one variable operating parameter of the laser source or determining the value of each of the plurality of base settings of the at least one variable operating parameter selected in the one or more iterations of the first process is based at least in part on the stored predetermined relationship.

19. The method of claim 14, wherein selecting the base setting from the plurality of base settings in the one or more iterations of the first process is based at least in part on the characteristic of the target in response to the laser light produced by the laser source and incident on the target in accordance with the value of the each of the plurality of base settings.

20. The method of claim 14, wherein selecting the optimized setting from the plurality of optimized settings in the one or more iterations of the second process is based at least in part on the determined characteristic of the target in response to the laser light produced by the laser source and incident on the target in accordance with the value of the each of the plurality of optimized settings.
